(19) United States
(12) Patent Application Publication
(10) Pub. No.: US 2015/0370984 A1
(43) Pub. Date: Dec. 24, 2015

(54) SYSTEM FOR OUTPATIENT MONITORING OF VENTRICULAR ASSISTANCE DEVICE PATIENTS

(71) Applicant: QUALITY ASSURED SERVICES, INC., Orlando, FL (US)

(72) Inventors: Betty RUSSELL, Southport, NC (US); Terry JOHNSON, Maitland, FL (US); Michelle ENGLAND, Chuluota, FL (US); Jose RIVERA, Kissimmee, FL (US)

(21) Appl. No.: 14/764,546

(22) PCT Filed: Jan. 30, 2014
(86) PCT No.: PCT/US14/13857

§ 371 Date: Jul. 29, 2015

Related U.S. Application Data
(60) Provisional application No. 61/758,642, filed on Jan. 30, 2013.

(51) Int. Cl. G06F 19/00; A61M 1/10; A61M 1/12

(52) U.S. Cl. CPC G06F 19/3418; G06F 19/322; A61M 1/122; A61M 1/1086; A61M 1/1005; A61M 2205/3334; A61M 2205/3584; A61M 2205/18; A61M 2205/84

(57) ABSTRACT

The present invention provides systems and methods for outpatient VAD management. The present invention can assist individuals with an implanted VAD, whether indicated as a bridge to recovery or transplantation or as destination therapy, and their associated caregives in the management of lifestyle modifications and long-tem management issues associated with VAD use.

The present application claims priority to U.S. Provisional Application No. 61/758,642 filed Jan. 30, 2013, which is hereby incorporated in its entirety including all tables, figures, and claims.

### BACKGROUND OF THE INVENTION

The following discussion of the background of the invention is merely provided to aid the reader in understanding the invention and is not admitted to describe or constitute prior art to the present invention.

A ventricular assist device (VAD) is a mechanical pump that is used to partially or completely replace the function of a failing heart. VADs are designed to assist either the right (RVAD) or left (LVAD) ventricle, or both ventricles (BiVAD). VADs are distinguished from “artificial hearts,” which are intended to completely replace, and generally require the removal of, the patient's heart. VADs may find use for patients recovering from heart attacks or heart surgery, while others are intended for long term use (months to years and in some cases for life), typically for patients suffering from congestive heart failure.

Recent refinements in VADs have improved the devices substantially in terms of size, simplicity, and reliability, and their ability to support patient care. As a result, while VADs were initiall intended as a bridge to recovery, the devices are now being used in patients as a permanent form of therapy. This increased use has required that VAD patients be monitored and supported on an outpatient basis.

### BRIEF SUMMARY OF THE INVENTION

The present invention provides systems and methods for outpatient VAD management. The present invention can assist individuals with an implanted VAD, whether indicated as a bridge to recovery or transplantation or as destination therapy, manage the lifestyle modifications and long-term management issues associated with VAD use. Additionally, the present invention can provide medical care providers with important data on VAD patients depending on these providers for improving their quality of life and care.

In a first aspect, the invention relates to computer-implemented systems for management and support of a plurality of ventricular assist device (VAD) patients on an outpatient basis. These systems comprise:
a plurality of first remote computers configured to be used by the plurality of VAD patients;
one or more second remote computers configured to be used by one or more caregivers supporting the care of the plurality of VAD patients;
a patient data server operably connected to the plurality of first remote computers and the one or more second remote computers, the patient data server comprising:
(i) patient-specific data file for each VAD patient, the patient-specific data file comprising patient identification data, medical data, physiological measurement data measured on an outpatient basis, and VAD data corresponding to each VAD patient,
(ii) a first executable software code configured to render a patient-directed interface on a first remote computer in response to entry of an unlock code specific to each patient on the first remote computer,
(iii) a second executable software code configured to render a caregiver-directed interface on a second remote computer in response to entry of an unlock code specific to a caregiver on the second remote computer,
(iv) a third executable code configured to receive data for each VAD patient from the plurality of first remote computers and store the data in the corresponding patient-specific data file for each VAD patient,
(v) a fourth executable code configured to process the data received for each VAD patient from the plurality of first remote computers and generate an alert viewable by the one or more caregivers when the processing indicates a data value out of a predefined range for the data.

The data received for each VAD patient can comprise both routine medical history information, as well as information specific to the patient's VAD use. By way of example only, the data may comprise patient identification data such as one or more of a patient name, a gender, a date of birth, and/or a mailing address; medical data such as one or more of a baseline weight measurement, a baseline blood pressure measurement, a baseline pulse rate, a baseline blood glucose concentration, a baseline prothrombin time measurement, a baseline international normalized ratio measurement, a medical history, and/or a medication usage history; physiological measurement data such as one or more of a series of daily weight measurements, a series of daily blood pressure measurements, a series of daily pulse rates, a series of daily blood glucose concentrations, a series of daily prothrombin time measurements, and/or a series of daily baseline international normalized ratio measurements; VAD data such as one or more of a reason for VAD use, a type of VAD, a VAD implantation date, a VAD flow rate, a VAD flow volume, a VAD speed, a VAD power level, and/or a VAD pulsatility index.

In certain embodiments, the computer-implemented system of one of claims 1-5, wherein the first, second, third, and fourth executable computer codes are implemented as a single program product. In other embodiments, these executable computer codes may be implemented as a plurality of separate programs. The patient-directed interface may be rendered in a web browser application operating on the first remote computer. In certain embodiments, the patient-directed interface is accessed via a web site to which the patients are enrolled in a secure fashion and which is viewed via a web browser or other client-side program that resides on the patient's local computer to facilitate the reading and or viewing or pages or presentations served via the Internet or Intranet.

In a similar fashion, the caregiver-directed interface may be rendered in a web browser application operating on the second remote computer. In certain embodiments, the caregiver-directed interface is accessed via a web site to which each authorized caregiver is enrolled in a secure fashion and which is viewed via a web browser or other client-side program that resides on the caregiver's local computer to facilitate the reading and or viewing or pages or presentations served via the Internet, Intranet, or virtual private network.

One or more data servers can function as a file server for the computer-implemented system and/or can include one or more of the files (e.g., application code, data files, etc.) necessary to implement methods of the invention incorporated by an application running on a user computer and/or another server. Alternatively, as those skilled in the art will appreciate, a file server can include all necessary files, allowing such an application to be invoked remotely by a user computer and/or server. It should be noted that the functions described with respect to various servers herein (e.g., application server, database server, web server, file server, etc.) can be performed by a single server and/or a plurality of specialized servers, depending on implementation-specific needs and parameters.

Preferably the system comprises one or more relational databases containing the patient-specific data in patient specific files. The relational databases may be controlled and managed by database software such as Microsoft SQL Server 7 by Microsoft Inc.

In practice, a sign-in page authenticates the identity of a patient or caregiver wishing to access the computer-implemented system. Upon authentication, the member may be provided with access to his or her home page. Patient home pages are preferably personalized to emphasize the information that is most relevant to the specific patient, while caregiver home pages preferably provide access to a group of patients being monitored by that caregiver, with an option to “drill down” for more detail on specific patients. Thus, in certain embodiments, the caregiver-directed interface comprises a first information display which provides simultaneous viewing of a subset or summary of data from the patient-specific data file for each of the VAD patients and a second information display which displays data from the patient-specific data file for a specified VAD patient at an increased level of detail. The caregiver-directed interface may further comprise an enrollment function for entering new VAD patients into the patient data server.

In order to serve an “urgent monitoring” function, the computer-implemented system may be configured to generate an alert for a specified VAD patient if processing the data for the specified VAD patient indicates non-compliance with a particular activity which is being monitored by the caregiver. By way of example only, the fourth executable code may be configured to generate an alert for a specified VAD patient if processing the data for the specified VAD patient indicates non-compliance with a medically indicated activity routine, such as a defined testing routine, an exercise routine, a medication routine, etc. The computer-implemented system may also be configured to generate an alert for a specified VAD patient if processing the data for the specified VAD patient indicates a variance in physiological measurement data such as one or more of a series of daily weight measurements, a series of daily blood pressure measurements, a series of daily pulse rates, a series of daily blood glucose concentrations, a series of daily prothrombin time measurements, and/or a series of daily baseline international normalized ratio measurements. In addition, the computer-implemented system may also be configured to generate an alert for a specified VAD patient if processing the data for the specified VAD patient indicates a variance in VAD function.

### BRIEF DESCRIPTION OF THE FIGURES

FIG. 1 depicts exemplary initial setup activities for an enrollment function for entering new VAD patients into the patient data server into a computer-implemented system of the invention, referred to as “VADWatch.”

FIG. 2 depicts exemplary primary monitoring activities of the VADWatch system.

FIG. 3 depicts recovery and termination monitoring activities of the VADWatch system.

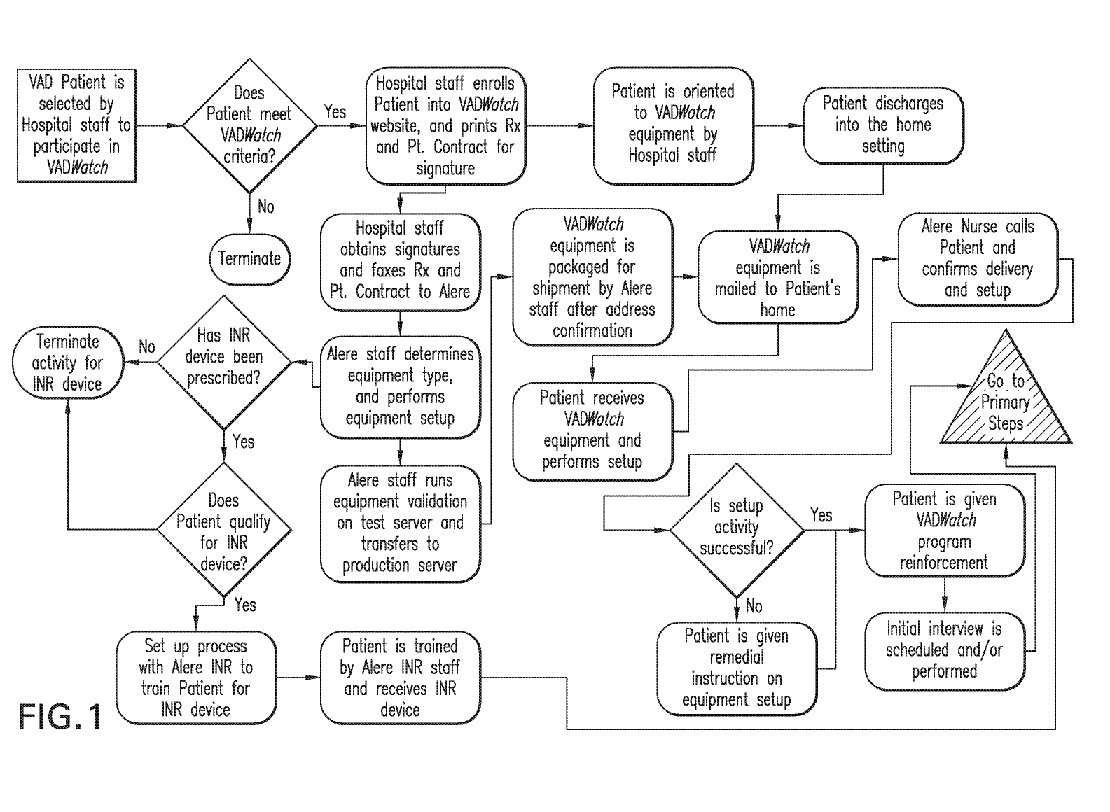

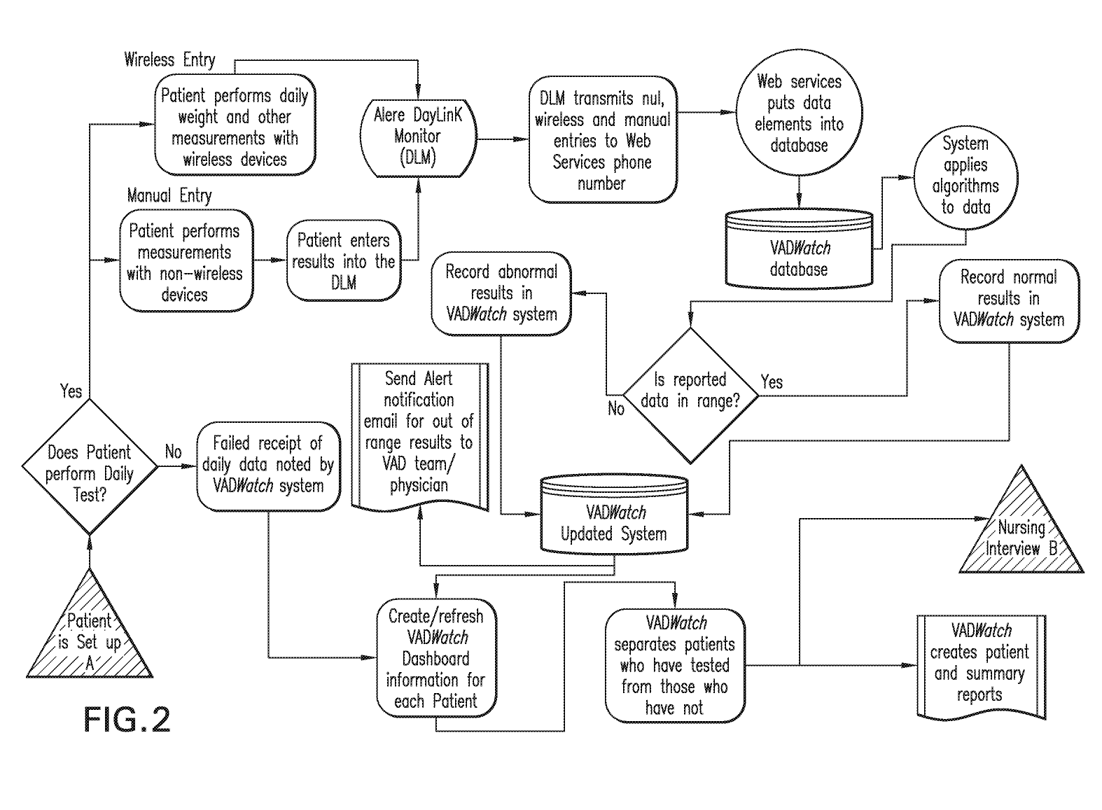

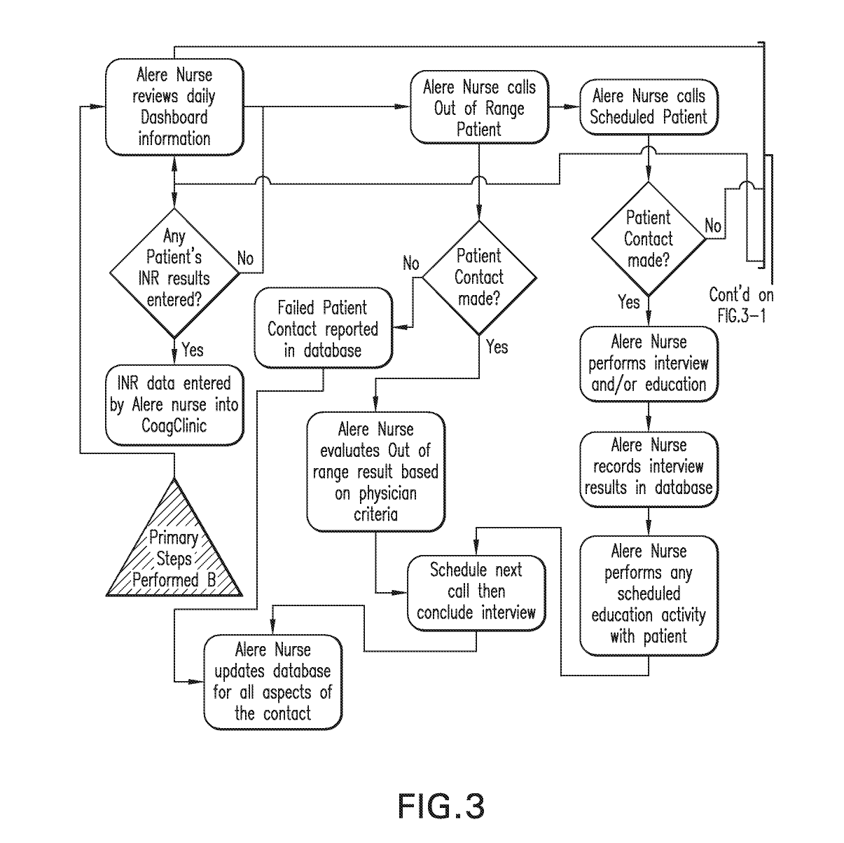

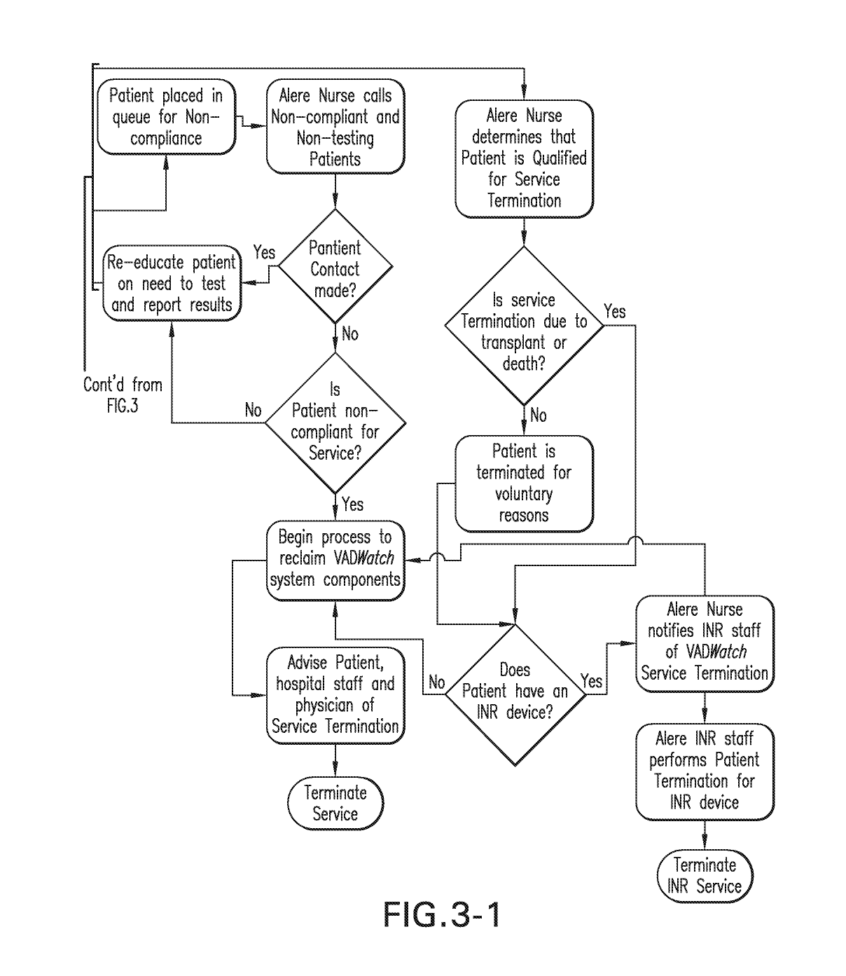

### DETAILED DESCRIPTION OF THE INVENTION

The present invention relates to computer-implemented systems for monitoring and care of VAD patients.

VADs used in the outpatient setting are surgically implanted devices connected to the heart by an inflow cannula that decompresses the ventricular cavity and an outflow cannula that returns blood to either the ascending aorta or the main pulmonary artery. The pumping chamber of the VAD is implanted distal to the heart (e.g., in a pre-peritoneal or intra-abdominal position or may be situated in a paracorporeal position outside the body). Smaller devices are being developed for thoracic implantation, some with outflow to the descending aorta. A percutaneous set of control and power wires connects the device to an external portable controller and a power supply that may be worn around the waist, carried in a shoulder bag, or contained within a small bedside monitor.

Before discharge, a VAD patient and his or her caregivers must be comfortable and competent to assume responsibility for daily monitoring, device maintenance, and independent performance of activities of daily living. This transition can be eased through the use of the systems for monitoring and care of VAD patients described herein, which can provide those involved with a strong sense of collaboration in the management of the patient.

VADs can be generally divided into “first generation” and “second generation” designs. First generation devices include those which pump in a plusatile fashion by compression of a chamber; these include the HeartMate® XVE, Novacor®, Thoratec PVAD or IVAD, Abiomal 5000, and LionHeart devices. Second generation devices include those which provide continuous flow using axial or centrifugal-type pumps; these include the HeartMate II®, Jarvik 2000, Incor, VentrAssist, Dura Heart®, HeartWare®, HeartQuest, and MTIHeart LVAD devices.

VADs have programmable functions including mode of operation, device rate, drive pressure, vacuum pressure, and duration of systole for the pneumatic pump. In certain cases, these controls, such as rotary speed for continuous flow pumps, are specific to pump type. Thus, the present systems for monitoring and care of VAD patients is preferably both “device-insensitive,” in that it can operate with VADs from a variety of manufacturers, and “device-sensitive,” in that it can include information about a particular patient's VAD type and assist the clinical team in monitoring the VAD-specific issues which arise therefrom.

The computer-implemented systems operate in conjunction with a variety of measurements and routines which may be prescribed or recommended to the VAD patient, and may advantageously used to monitor compliance with a schedule of activities to be preformed on an outpatient basis. These activities may be performed by the patient, by a home heathcare worker, or by a combination thereof. The following summary of such activities are not meant to be limiting.

Physical Exercise

Once discharged, a schedule of physical exercise may be prescribed or recommended to the VAD patient. If deconditioned, patients should be sent to an outpatient cardiac rehabilitation program to help them work on gaining strength and improving their endurance and energy capacity.

Nutrition

The nutritional status of a VAD patient should be carefully monitored, as nutritional status is important to the perioperative management of VAD patients. Cachexia, defined as a body mass index <21 kg/m2 in males and <19 kg/m2 in females, is a strong independent predictor of mortality, along with other markers of poor nutritional status, including low serum levels of albumin, prealbumin, and total protein; reduced absolute lymphocyte count; and elevated C-reactive protein. Adequate nutritional support reduces the risk of postoperative infection and improves functional recovery. Additionally, weight loss or weight gain should be monitored for purposes of maintaining optimal fluid balance.

Blood Pressure and Vital Signs

Hemodynamic measurements in the context of the VAD patient includes certain special considerations. Blood pressure needs to be carefully measured because systemic hypertension has been seen in both ischemic and nonischemic cardiomyopathy VAD patients, particularly those with with pulsatile VADs. Obtaining systolic pressure by radial artery palpation is preferred over brachial artery auscultation for conventional pulsatile technology because the VAD device can transmit sounds that can be confused with Korotkoff sounds. With axial flow pumps, no audible aortic valve closure sound occurs because there is no or minimal pulse pressure to be measured by palpation. When defining the blood pressure, it is recommended that a Doppler flow probe be used to help define the blood pressure. Cardiac output may be monitored by mixed venous oxygen saturation measurement.

Anticoagulation and Antiplatelet Therapy

Anticoagulation or antiplatelet therapy is a central component of outpatient management because thromboembolism is associated with all devices. International normalized ratios of 1.5 to 2.5 are currently targeted for pneumatically driven pulsatile devices. In patients with a continuous flow pump, some VAD centers are now recommending a lower international normalized ratio of 1.7 to 2.3. VAD patients should be monitored closely to minimize the risk of a gastrointestinal or intracranial bleed or severe epistaxis. Adjustments to the anticoagulant regimen may be directed by the physician in conjunction with a patient's specific needs and the physician's own practicing patterns.

Mental Status

Mental status examination is an important part of the clinical assessment process and may be used to assess potential neurologic events subsequent to VAD implantation. While a questionnaire is not an ideal method of assessing mental status, it can provide a valuable adjunct between visits with a clinician. The patient-specific interface of the system described herein may include such a set of questions to assess accuracy and pace of responses.

Status of VAD

Device malfunction is an important cause of morbidity and mortality in patients living with VADs, especially with the prolonged support required for both bridge to transplantation and destination therapy. Complications can arise in any component from the portable drive/system controller that controls and powers the device to the inflow and outflow cannulae, valves, batteries, and the VAD itself. All devices have system controllers and monitors which can be integrated with the management system of the present invention. Alerts should be used in conjunction with clinical, laboratory, and imaging data to diagnose suspected device malfunction. For troubleshooting, systematic catheter-, angiography-, fluoroscopy-, and echocardiography-based protocols have been developed to help diagnose common malfunctions.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. A computer-implemented system for management and support of a plurality of ventricular assist device (VAD) patients on an outpatient basis, comprising:
a plurality of first remote computers configured to be used by the plurality of VAD patients;
one or more second remote computers configured to be used by one or more caregivers supporting the care of the plurality of VAD patients;
a patient data server operably connected to the plurality of first remote computers and the one or more second remote computers, the patient data server comprising:
(i) patient-specific data file for each VAD patient, the patient-specific data file comprising patient identification data, medical data, physiological measurement data measured on an outpatient basis, and VAD data corresponding to each VAD patient,
(ii) a first executable software code configured to render a patient-directed interface on a first remote computer in response to entry of an unlock code specific to each patient on the first remote computer,
(iii) a second executable software code configured to render a caregiver-directed interface on a second remote computer in response to entry of an unlock code specific to a caregiver on the second remote computer,
(iv) a third executable code configured to receive data for each VAD patient from the plurality of first remote computers and store the data in the corresponding patient-specific data file for each VAD patient,
(v) a fourth executable code configured to process the data received for each VAD patient from the plurality of first remote computers and generate an alert viewable by the one or more caregivers when the processing indicates a data value out of a predefined range for the data.

2. The computer-implemented system of claim 1, wherein the patient identification data comprises one or more of a patient name, a gender, a date of birth, and a mailing address.

3. The computer-implemented system of claim 1, wherein the medical data comprises one or more of a baseline weight measurement, a baseline blood pressure measurement, a baseline pulse rate, a baseline blood glucose concentration, a baseline prothrombin time measurement, a baseline international normalized ratio measurement, a medical history, and a medication usage history.

4. The computer-implemented system of claim 1, wherein the physiological measurement data comprises one or more of a series of daily weight measurements, a series of daily blood pressure measurements, a series of daily pulse rates, a series of daily blood glucose concentrations, a series of daily prothrombin time measurements, and a series of daily baseline international normalized ratio measurements.

5. The computer-implemented system of claim 1, wherein the VAD data comprises one or more of a reason for VAD use, a type of VAD, a VAD implantation date, a VAD flow rate, a VAD flow volume, a VAD speed, a VAD power level, and a VAD pulsatility index.

6. The computer-implemented system of claim 1, wherein the first, second, third, and fourth executable computer codes are implemented as a single program product.

7. The computer-implemented system of claim 1, wherein the patient-directed interface is rendered in a web browser application operating on the first remote computer.

8. The computer-implemented system of claim 1, wherein the caregiver-directed interface is rendered in a web browser application operating on the second remote computer.

9. The computer-implemented system of claim 1, wherein the patient data server stores each patient-specific data file in an SQL database.

10. The computer-implemented system of claim 1, wherein the patient data server is operably connected to the plurality of first remote computers and the one or more second remote computers via an internet connection.

11. The computer-implemented system of claim 1, wherein wherein the patient data server is operably connected to the plurality of first remote computers and the one or more second remote computers via a virtual private network.

12. The computer-implemented system of claim 1, wherein the caregiver-directed interface comprises a first information display which provides simultaneous viewing of a subset or summary of data from the patient-specific data file for each of the VAD patients and a second information display which displays data from the patient-specific data file for a specified VAD patient at an increased level of detail.

13. The computer-implemented system of claim 12, wherein the caregiver-directed interface further comprising an enrollment function for entering new VAD patients into the patient data server.

14. The computer-implemented system of claim 1, wherein the fourth executable code generates an alert for a specified VAD patient if processing the data for the specified VAD patient indicates non-compliance with a testing routine.

15. The computer-implemented system of claim 1, wherein the fourth executable code generates an alert for a specified VAD patient if processing the data for the specified VAD patient indicates non-compliance with a medically indicated activity routine.
